(19) United States
(12) Patent Application Publication
(10) Pub. No.: US 2026/0026750 A1
(43) Pub. Date: Jan. 29, 2026

(54) IMPLANTABLE MEDICAL DEVICE WITH PATTERNED ELECTRODES

(71) Applicant: Cardiac Pacemakers, Inc., St Paul, MN (US)

(72) Inventors: Thomas John Herbst, Coon Rapids, MN (US); Jean M. Bobgan, Maple Grove, MN (US); Keith R. Maile, New Brighton, MN (US)

(21) Appl. No.: 19/278,049

(22) Filed: Jul. 23, 2025

Related U.S. Application Data
(60) Provisional application No. 63/676,083, filed on Jul. 26, 2024.

(51) Int. Cl. A61B 5/00; A61B 5/29; A61N 1/05; A61N 1/362; A61N 1/375; A61N 1/39

(52) U.S. Cl. CPC A61B 5/686; A61B 5/29; A61N 1/0587; A61N 1/362; A61N 1/3756; A61N 1/3956; A61B 2560/0468; A61B 2562/0209; A61B 2562/125

(57) ABSTRACT

An implantable medical device (IMD) including a housing having an outer surface and extending between a first housing end and a second housing end. The IMD includes an electrode coupled to the housing and positioned at or adjacent to the first housing end, and the electrode has an electrode surface. The IMD includes a surface area on the electrode surface or adjacent to the electrode. The surface area includes micropores and channels interconnecting the micropores.

### CROSS REFERENCE TO RELATED APPLICATION

This application claims priority to Provisional Application No. 63/676,083, filed Jul. 26, 2024, which is herein incorporated by reference in its entirety.

### TECHNICAL FIELD

Instances of the present disclosure relate to medical devices and systems for sensing physiological parameters and/or delivering therapy. More specifically, instances of the disclosure relate to implantable medical devices with mechanical features for encouraging tissue-to-electrode and/or tissue-to-housing integration.

### BACKGROUND

Certain implantable medical devices include one or more electrodes for assisting with sensing physiological parameters such as cardiac activation signals.

### SUMMARY

In Example 1, an implantable medical device (IMD) including a housing having an outer surface and extending between a first housing end and a second housing end. The IMD includes an electrode coupled to the housing and positioned at or adjacent to the first housing end, and the electrode has an electrode surface. The IMD includes a surface area on the electrode surface or adjacent to the electrode. The surface area includes micropores and channels interconnecting the micropores.

In Example 2, the IMD of Example 1, wherein the surface area is on the electrode surface.

In Example 3, the IMD of Example 2, wherein the electrode is a face electrode.

In Example 4, the IMD of Example 2, wherein the electrode is an end electrode.

In Example 5, the IMD of Example 1, wherein the electrode is a first electrode, the IMD further comprising a second electrode that includes a second set of micropores and a second set of channels interconnecting the second set of micropores.

In Example 6, the IMD of Example 5, wherein the first electrode is an end electrode, wherein the second electrode is a face electrode.

In Example 7, the IMD of Example 5, wherein the first electrode is a first end electrode, wherein the second electrode is a second electrode.

In Example 8, the IMD of any of Examples 1-7, wherein the micropores include a first micropore and a second micropore adjacent to each other, wherein one of the channels extends between the first micropore and the second micropore.

In Example 9, the IMD of any of Examples 1-8, wherein the micropores have an opening that is 50-200 micrometers.

In Example 10, the IMD of any of Examples 1-9, wherein the micropores have a depth of 100-500 micrometers.

In Example 11, the IMD of any of Examples 1-10, wherein the channels have an opening of 30-60 micrometers.

In Example 12, the IMD of any of Examples 1-11, wherein the electrode is directly coupled to the housing.

In Example 13, the IMD of any of Examples 1-12, wherein the micropores have circular-shaped openings.

In Example 14, the IMD of any of Examples 1-12, wherein the micropores have square-shaped openings.

In Example 15, the IMD of Examples 1-14, further comprising protrusions disposed on the outer surface of the housing, wherein the protrusions have holes extending through the protrusions.

In Example 16, an IMD including a housing having an outer surface and extending between a first housing end and a second housing end. The IMD includes an electrode coupled to the housing and positioned at or adjacent to the first housing end, and the electrode has an electrode surface. The IMD includes a surface area on the electrode surface or adjacent to the electrode. The surface area includes micropores and channels interconnecting the micropores.

In Example 17, the IMD of Example 16, wherein the surface area is on the electrode surface.

In Example 19, the IMD of Example 17, wherein the electrode is an end electrode.

In Example 20, the IMD of Example 16, wherein the electrode is a first electrode, the IMD further comprising a second electrode that includes a second set of micropores and a second set of channels interconnecting the second set of micropores.

In Example 18, the IMD of Example 17, wherein the electrode is a face electrode.

In Example 21, the IMD of Example 20, wherein the first electrode is an end electrode, wherein the second electrode is a face electrode.

In Example 22, the IMD of Example 21, wherein the first electrode is a first end electrode, wherein the second electrode is a second electrode.

In Example 23, the IMD of Example 16, wherein the micropores include a first micropore and a second micropore adjacent to each other, wherein one of the channels extends between the first micropore and the second micropore.

In Example 24, the IMD of Example 16, wherein the micropores have an opening that is 50-200 micrometers.

In Example 25, the IMD of Example 16, wherein the micropores have a depth of 100-500 micrometers.

In Example 26, the IMD of Example 16, wherein the channels have an opening of 30-60 micrometers.

In Example 27, the IMD of Example 16, wherein the electrode is directly coupled to the housing.

In Example 28, the IMD of Example 16, wherein the micropores have circular-shaped openings.

In Example 29, the IMD of Example 16, wherein the micropores have square-shaped openings.

In Example 30, the IMD of Example 16, further comprising protrusions disposed on the outer surface of the housing, wherein the protrusions have holes extending through the protrusions.

In Example 31, the IMD of Example 16, wherein a majority of the micropores are connected to at least one other micropore via at least one of the channels.

In Example 32, the IMD of Example 16, wherein the micropores and the channels are shaped and sized for deposition of extracellular matrix proteins.

In Example 33, an IMD including a housing having an outer surface and extending between a first housing end and a second housing end. The IMD includes an electrode coupled to the housing and positioned at or adjacent to the first housing end, and the electrode has an electrode surface. The IMD includes a surface area on the electrode surface or adjacent to the electrode. The surface area includes micropores and channels interconnecting the micropores. The micropores have respective openings that are 50-200 micrometers, the micropores have respective depths of 100-500 micrometers, and the channels have respective diameters of 30-60 micrometers.

In Example 34, a method of making an IMD, the method including forming a housing having an outer surface and extending between a first housing end and a second housing end; disposing an electrode on the outer surface of the housing; and forming a microporous surface area on the electrode or adjacent to the electrode. The microporous surface area includes micropores and channels interconnecting the micropores.

In Example 35, the method of Example 34, wherein the micropores include a first micropore and a second micropore adjacent to each other, wherein one of the channels extends between the first micropore and the second micropore.

While multiple instances are disclosed, still other instances of the present disclosure will become apparent to those skilled in the art from the following detailed description, which shows and describes illustrative instances of the disclosure. Accordingly, the drawings and detailed description are to be regarded as illustrative in nature and not restrictive.

### BRIEF DESCRIPTION OF THE DRAWINGS

FIG. 1 is a schematic illustration depicting a patient monitoring system, in accordance with certain instances of the present disclosure.

FIG. 2A is a side view of an implantable medical device, in accordance with certain instances of the present disclosure.

FIG. 2B is a simplified schematic diagram of an implantable medical device, in accordance with certain instances of the present disclosure.

FIGS. 3A-3B are side views of an end electrode of an implantable medical device, in accordance with certain instances of the present disclosure.

FIG. 4 is a side view of an implantable medical device, in accordance with certain instances of the present disclosure.

FIGS. 5A-5F are perspective views of an electrode of an implantable medical device, in accordance with certain instances of the present disclosure.

FIG. 6 is a side view of a portion of an IMD being gripped by a portion of a medical forceps, in accordance with certain instances of the present disclosure.

FIG. 7 depicts a block diagram of a method of manufacturing an implantable medical device described herein, in accordance with certain instances of the present disclosure.

While the disclosed subject matter is amenable to various modifications and alternative forms, specific instances have been shown by way of example in the drawings and are described in detail below. The intention, however, is not to limit the disclosed subject matter to the particular instances described. On the contrary, the disclosed subject matter is intended to cover all modifications, equivalents, and alternatives falling within the scope of the disclosed subject matter as defined by the appended claims.

### DETAILED DESCRIPTION

After a medical device is implanted subcutaneously within a patient's body (e.g., in the patient's chest or abdomen), the surrounding tissue attempts to heal. The healing process may occur during a time window of 4-12 weeks. During the time window, the body may isolate the device and encapsulate it by fibroplasia (e.g., healing by fibrous encapsulation). This fibrous encapsulation can create a fibrous pocket over time which can isolate the device and negatively impact sensing features.

The inventors of the present disclosure have developed approaches that can promote cellular/tissue integration into the device and interrupt or otherwise deter the encapsulation process as the body heals after implantation. For example, modifying an outer surface of the medical device (e.g., a portion of an electrode and/or housing) to include a microporous surface area can create a tissue-to-electrode and/or a tissue-to-housing integrated interface. This reduces the risk of an anatomic boundary layer forming during the encapsulation process. Creating a tissue-to-electrode interface and/or a tissue-to-housing interface can improve electrical connectivity because the electrode(s) are better able to sense cardiac activation signals. In contrast, a poor tissue-to-electrode interface may cause fluctuating or poor measurements associated with the medical device (e.g., fluctuating or poor impedance measurements).

Certain instances of the present disclosure utilize one or more microporous surface areas on and/or adjacent to one or more electrodes on an outer surface of an implanted medical device. The microporous surface areas are designed to promote cellular/tissue integration with the one or more electrodes or other outer surfaces of the medical device.

### System

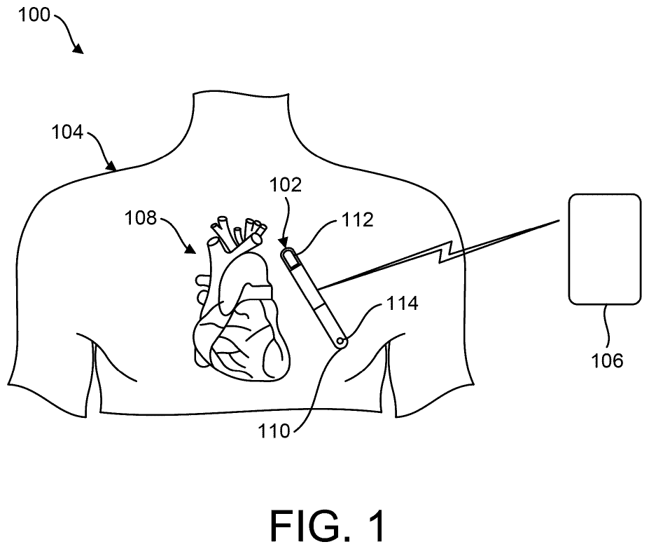

FIG. 1 is a schematic illustration of a system 100 including an implantable medical device (IMD) 102 implanted within a patient's body 104 and configured to communicate with a receiving device 106. The IMD 102 can be implanted subcutaneously within an implantation location or pocket in the patient's chest or abdomen and can be configured to monitor (e.g., sense and/or record) physiological parameters associated with the patient's heart 108. The IMD 102 can be an implantable cardiac monitor (e.g., an implantable diagnostic monitor, an implantable loop recorder) configured to record physiological parameters such as, for example, one or more cardiac activation signals, heart sounds, blood pressure measurements, oxygen saturations. Further, the IMD 102 can be configured to monitor physiological parameters that may include one or more signals indicative of a patient's physical activity level and/or metabolic level, such as an acceleration signal.

For purposes of illustration, and not of limitation, various instances of devices that may be used to record physiological parameters in accordance with the present disclosure are described herein in the context of IMDs that may be implanted under the skin in the chest region of a patient. However, the IMD 102 may include any type of IMD, any number of different components of an implantable system, and/or the like having a housing and being configured to be implanted in a patient's body 104. For example, the IMD 102 can include a control device, a monitoring device, a pacemaker, an implantable cardioverter defibrillator (ICD), a cardiac resynchronization therapy (CRT) device and/or the like, and may be an implantable medical device known in the art or later developed, for providing therapy and/or diagnostic data about the patient's body. In various instances, the IMD 102 can include both defibrillation and pacing/CRT capabilities (e.g., a CRT-D device).

As shown, the IMD 102 includes a housing 110 having multiple electrodes 112 and 114 coupled thereto (e.g., electrodes 112 and 114 directly mechanically coupled to the housing 110). As will be described in greater detail, the electrodes 112 and 114 are disposed at opposite ends or leading edges of the housing 110 along a longitudinal direction. In some instances, at least one of the electrodes is wrapped around a leading edge of the housing 110 to form an end electrode. Further, in some instances, at least one of the electrodes is provided with a microporous surface area on the electrode surface. The microporous surface area can include surface textures that are shaped and sized to encourage cellular/tissue in-growth on (and/or within) the microporous surface area after implantation of the IMD 102.

The IMD 102 can be configured to sense physiological parameters and record the sensed physiological parameters. For example, the IMD 102 may be configured to activate (e.g., periodically, continuously, upon detection of an event, and/or the like), record a specified amount of data (e.g., physiological parameters) in a memory, and communicate that recorded data to the receiving device 106. The receiving device 106 may be, for example, a programmer, controller, patient monitoring system, and/or the like. Although illustrated in FIG. 1 as an external device, the receiving device 106 can include an implantable device configured to communicate with the IMD 102 that can, for example, be a control device, another monitoring device, a pacemaker, an ICD, a CRT device, and/or the like.

The IMD 102 and the receiving device 106 can communicate through a wireless link. For example, as will be described in more detail below, the IMD 102 can include an antenna, which transmits and/or receives signals from the receiving device 106. The IMD 102 and the receiving device 106 can be communicatively coupled through a short-range communications link, such as Bluetooth, IEEE 802.11, and/or a proprietary wireless protocol. The communications link can facilitate uni-directional and/or bi-directional communication between the IMD 102 and the receiving device 106. Data and/or control signals can be transmitted between the IMD 102 and the receiving device 106 to coordinate the functions of the IMD 102 and/or the receiving device 106. The patient data can be downloaded from one or more of the IMD 102 and the receiving device 106 periodically or on command. The physician and/or the patient may communicate with the IMD 102 and the receiving device 106, for example, to acquire patient data or to initiate, terminate, or modify recording and/or therapy.

### Medical Device

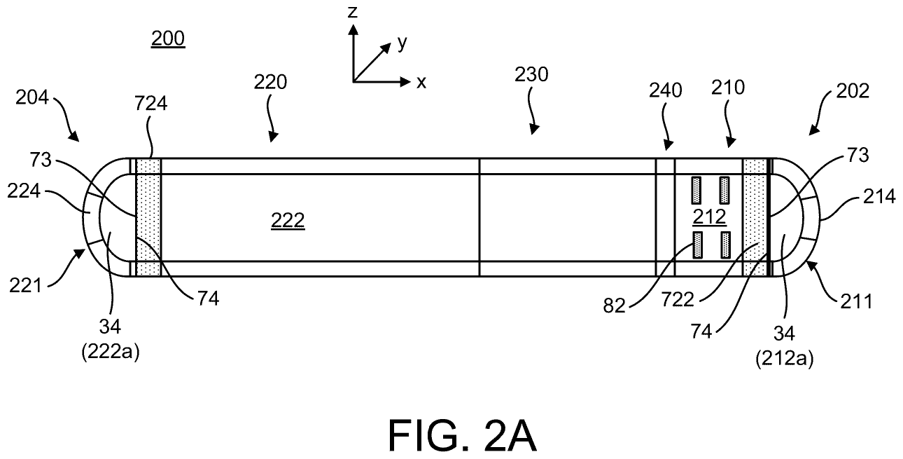

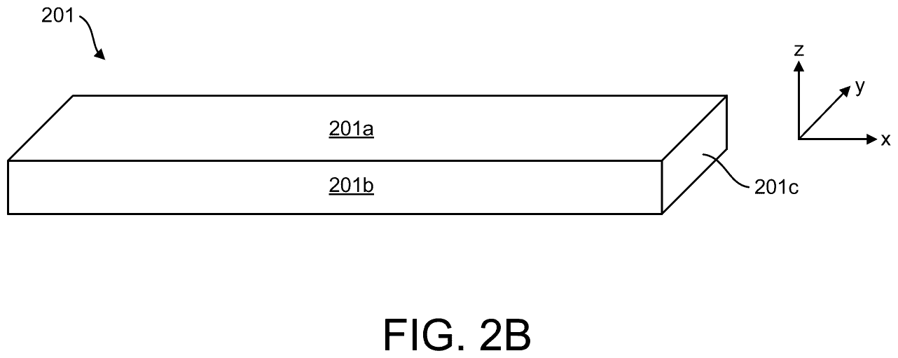

FIG. 2A is a side view of an implantable medical device 200 (hereinafter “IMD 200” for brevity). The IMD 200 may be, or may be similar to, the IMD 102 depicted in FIG. 1 and may be used in the system 100 of FIG. 1.

The IMD 200 includes an external housing that extends between a first end 202 and a second end 204. FIG. 2B is a simplified schematic diagram of an example external housing 201. As shown, the external housing 201 has a general shape of elongate strip with a length, a width, and a thickness along the x, y and z axes, respectively. The length of the external housing 201 can be generally greater than the width, and greater than the thickness. The external housing 201 includes two opposite major surfaces or faces 201a substantially in the x-y plane, two opposite side surfaces or sides 201b substantially in the x-z plane, and two opposite end surfaces or leading edges 201c substantially in the y-z plane. While a cross-sectional rectangular shape is show in the example schematic diagram of FIG. 2B, it is to be understood that the external housing 201 may have a regular or irregular cross-sectional shape such as, for example, a rectangular shape, an oval shape, a generally round shape, a trapezoid shape, and the like. The adjacent surfaces 201a, 201b, and 201c can be connected by a transition portion such as a curved surface, a chamfer, and the like.

In the example of FIG. 2A, the IMD 200 includes a first housing section 210, a second housing section 220, a third housing section 230, and a fourth housing section 240. The first housing section 210 is located adjacent to the first end 202 and includes a first major surface 212 (e.g., a portion of each of the opposite major surfaces 201a of FIG. 2B adjacent to the first end 202) and a first end surface or leading edge 214 (e.g., one of the end surfaces 201c of FIG. 2B). The second housing section 220 is located adjacent to the second end 204 and includes a second major surface 222 (e.g., a portion of each of the opposite major surfaces 201a of FIG. 2B adjacent to the second end 204) and a second end surface or leading edge 224 (another of the end surfaces 201c of FIG. 2B). Each of the housing sections can be separate components that are assembled together during manufacturing to create the external housing of the IMD 200. When assembled together, the housing sections can create a hermetically sealed enclosure. Although four separate housing sections are shown in FIG. 2A, additional or fewer separate sections can be used to create the IMD 200.

As shown, the IMD 200 further includes a first electrode 211 disposed at the first end 202, and a second electrode 221 disposed at the second end 204. The first electrode 211 is configured to wrap around the first end surface or leading edge 214 at the first end 202 and can be referred to as a first end electrode. The second electrode 221 is configured to wrap around the second end surface or leading edge 214 at the second end 204 and is referred to as a second end electrode.

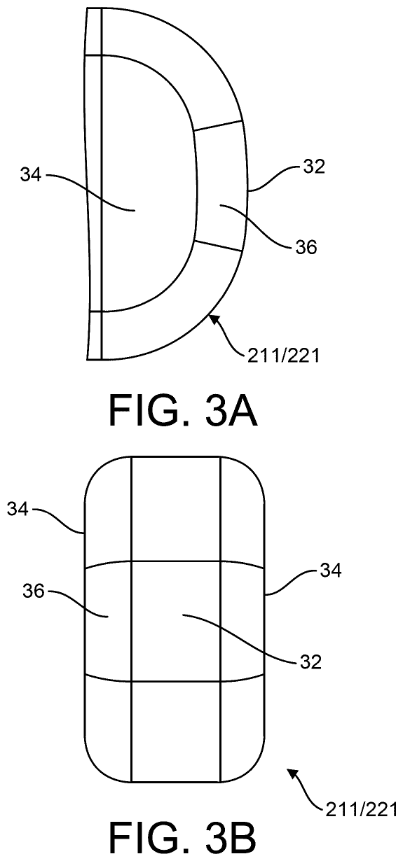

FIGS. 3A and 3B illustrate side views of an example first or second end electrode. The end electrodes can include a curved edge electrode portion 32, a face electrode portion 34, and a transition portion 36 to connect the curved edge electrode portion 32 to the face electrode portion 34. The curved edge electrode portion 32 is configured to form at least a portion of the first or second end surface (e.g., the end surface 214 or 224 of FIG. 2A, or the end surface 201c of FIG. 2B). The face electrode portion 34 is configured to cover and/or form at least a portion of the first or second major surface 212 or 214 of the first or second housing section 210 or 220 adjacent to the respective ends 202 and 204 (FIG. 2A). For example, as shown in FIG. 2A, the first major surface 212 of the first housing section 210 has an end portion 212a, which can be covered by the face electrode portion 34 of the first end electrode 211, and the second major surface 222 of the second housing section 220 has an end portion 222a, which can be covered by the face electrode portion 34 of the second end electrode 221. The end electrodes are shaped to fit over the body of the housing of an IMD. For example, the internal surface of the end electrodes can form a concave shape, and the internal surface can be directly coupled to the body of an IMD.

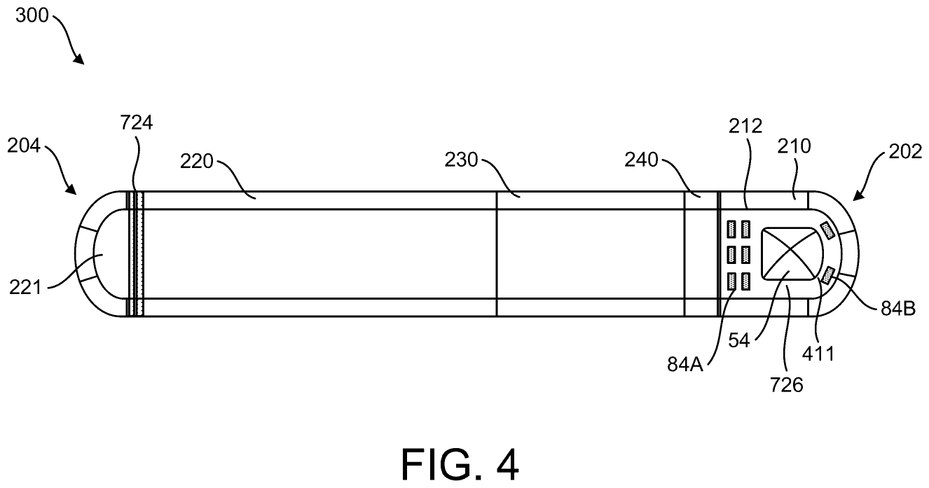

FIG. 4 shows an IMD 300 with a different electrode configuration. In the example of FIG. 4, the IMD 300 includes an end electrode 221 disposed at the first end 204 and a face electrode 411 coupled to the first major surface 212 of the first housing section 210 near the first end 202. The end electrode 221 wraps around the second end surface or leading edge to form the second end 204. The face electrode 411 includes an electrode surface 54.

As described in more detail below, the IMDs can include one or more of the electrodes (such as those described herein) with a microporous surface area (e.g., an area with multiple micropores) on the electrode surface to encourage cellular/tissue in-growth with the microporous surface area after implantation of the IMD. Such in-growth can create a stable tissue-to-electrode contacting interface and enhance the mechanical and electrical stabilization of the IMD within the implantation location, which in turn improves performance of the IMD. In certain instances, to improve tissue-to-electrode contact, one or more microporous surface areas are positioned adjacent to one or more electrodes and on an outer surface of an IMD to also encourage cellular/tissue in-growth with the microporous surface area.

In certain instances, the microporous surface areas described herein include one or more microporous surface features or microporous surface textures. Such surface feature or surface texture may include, for example, sets of micropores formed by protrusions (e.g., sets of protrusions each shaped as a line, a bar, a grid pattern, an island, a mesh structure, and the like), and/or one or more sets of depressions (e.g., sets of depressions each shaped as an opening, a pore, a hole, a pit, a well, a channel, and the like). In certain instances, the micropores are 1,000 micrometers or less in diameter, length, and/or depth.

In some instances, the microporous surface areas described herein permits cellular-level tissue attachment and migration into the porous interstices. The microporous surface features or microporous surface textures within the microporous surface areas can facilitate, for example, mesenchymal cell migration and ingrowth (e.g., fibroblasts, smooth muscle cells, myofibroblasts) and/or collagen fiber deposition into and onto the microporous surface areas, which can provide a cellular-level integration/fixation of the subcutaneous tissue with portions of one or more electrodes and/or housing of an IMD. The microporosity first allows migration of inflammatory (e.g., neutrophils, macrophages, lymphocytes) egress of blood component proteins such as fibrin, fibronectin, albumen, edema fluid-which can stimulate the healing integration of mesenchymal cells into and onto these microporous surface areas. The type of tissue features that attach to one or more portions of the porous surface areas may include, for example, fibroblasts, myofibroblasts, smooth muscle cells, various neovascularization events, coupled with collagen and/or proteoglycan extracellular matrix deposition as a stabilizing protein matrix. In the absence of the microporous surface area described herein, an IMD may become isolated from the surrounding subcutaneous tissue by the formation of a fibrous capsule-which effectively seals the IMD in a fibrous pocket.

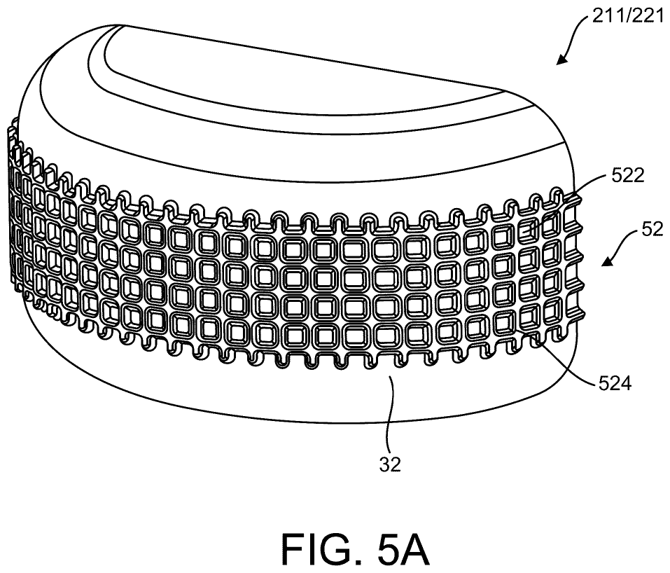

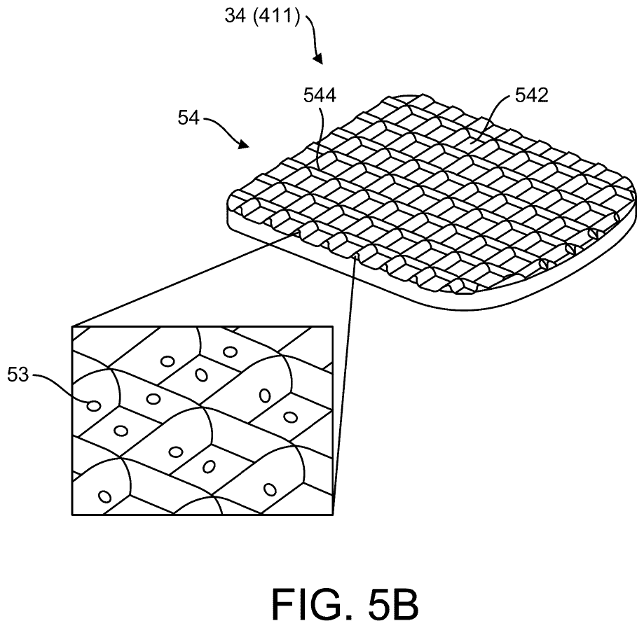

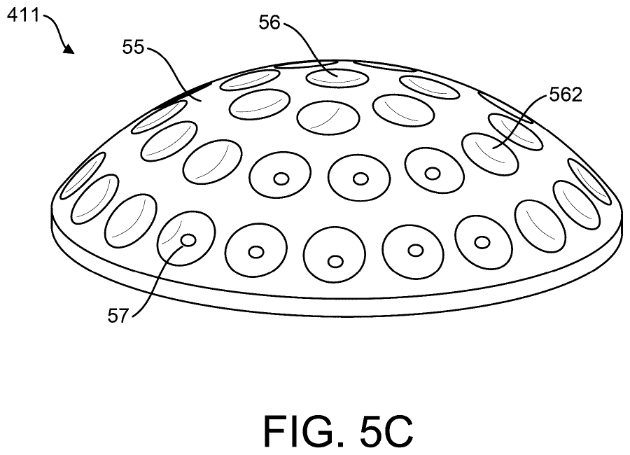

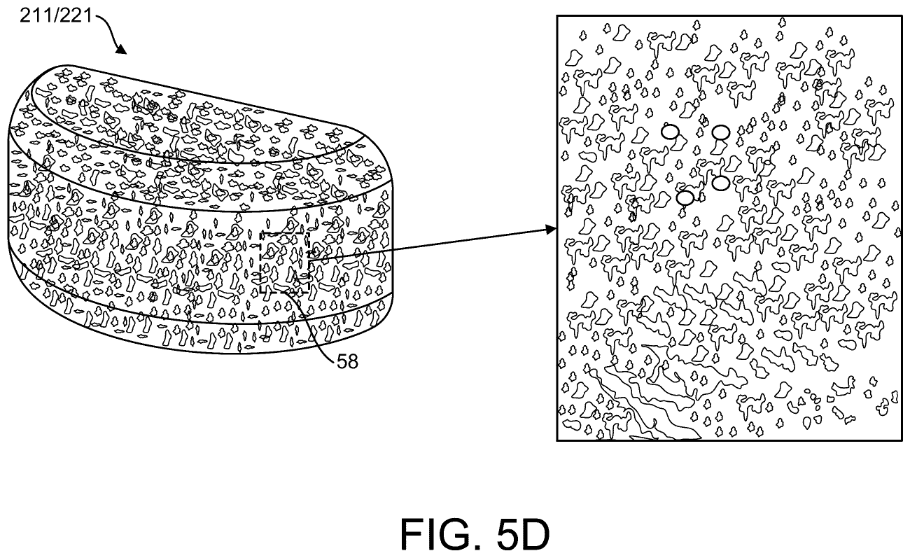

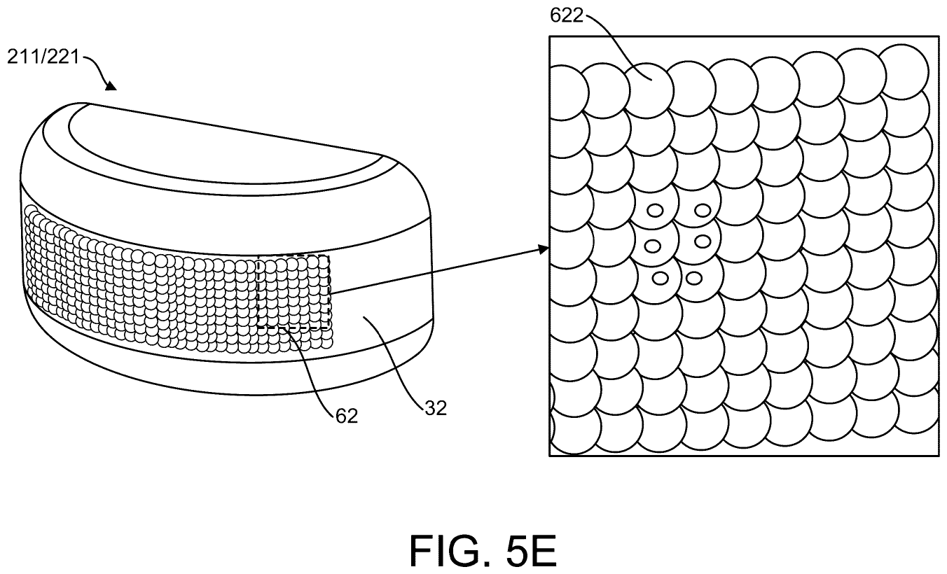

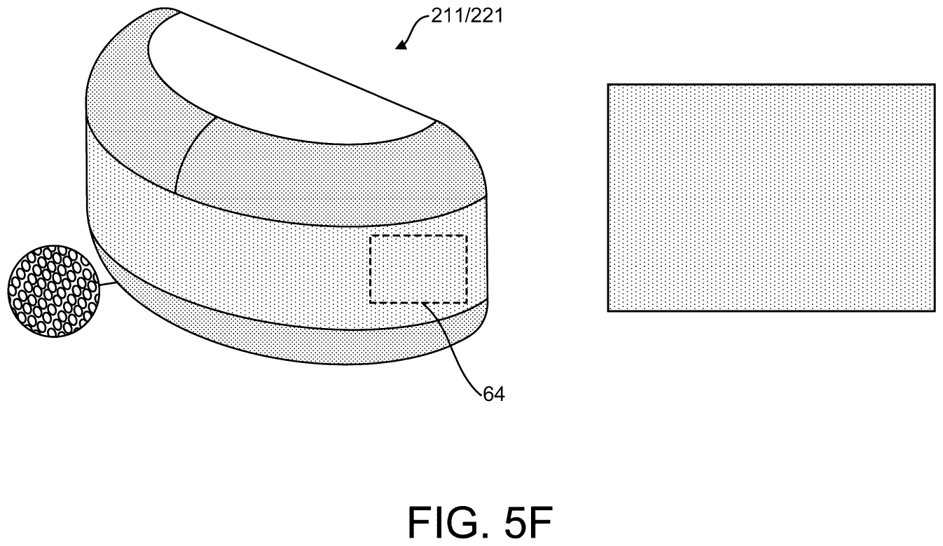

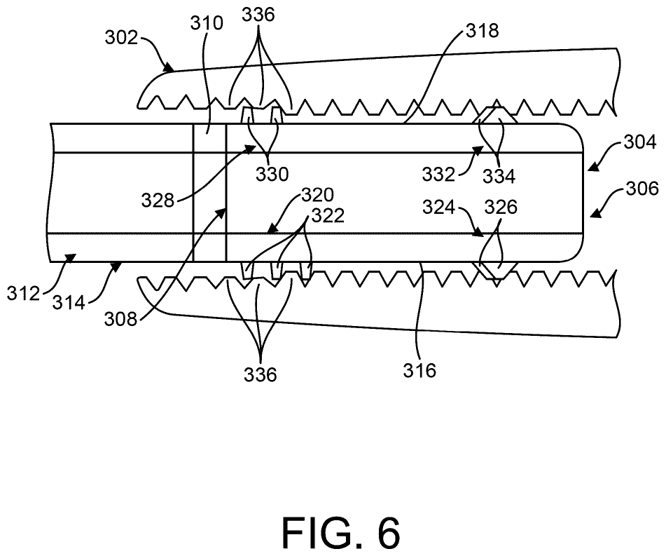

FIGS. 5A-5F illustrate various examples of a microporous surface area formed on a surface of an electrode coupled to the housing of an IMD. Electrodes can be formed of electrically conductive materials such as materials that contain titanium or a nickel-cobalt base alloy (e.g., MP35N®). These conductive materials can form a base substrate of the electrode. In certain instances, the base substrate is at least partially covered by a coating. The coating can comprise a material such as titanium nitride, iridium oxide, and the like, that is textured as described herein to promote cellular/tissue integration. In other instances, the coating can comprise a polymer mesh that is textured as described herein to promote cellular/tissue integration.

At least a portion of the electrode can be made to form a microporous surface area which allows cellular/tissue growth into and onto the microporous surface area after implantation of the IMD. The microporous surface area(s) can be formed on an electrode by processes including, for example, chemical etching, laser texturing, metal additive manufacturing (e.g., 3D-metal printing), metal stamping, indenting, extrusion, die forming, forging, and the like. Examples of microporous surface areas and their features are described in detail below.

In the example of FIG. 5A, a microporous surface texture 52 is provided on the curved edge electrode portion 32 (FIGS. 3A-3B) of an end electrode (e.g., the end electrodes of FIGS. 2A and 4). The microporous surface texture 52 includes an array of micropores (referred to as the “wells 522” because of their shape) in a mesh-like structure 524. The mesh structure 524 is formed by crossed bars/walls on the electrode surface 32 to define the square-shaped wells 522. It is to be understood that the wells 522 can have regular or irregular shapes such as, for example, a polygonal shape, a circular shape, an oval shape, and the like with a 50-200 micrometer opening (e.g., diameter of the opening or length of the opening). As such, the micropores can have an opening with an area of 2,500-40,000 μm² (e.g., 5,000-35,000; 10,000-30,000; 15,000-25,000 μm²).

In some instances, the wells 522 may be formed on the electrode surface 32. In some instances, the wells 522 may be formed at least partially into the electrode surface 32. The wells 522 may have a depth in the range, for example, from 100-500 micrometers. As such, the micropores can have a volume of 0.00025-0.2 mm³ (0.001-0.15, 0.0015-0.10 mm³).

As will be described more in connection with FIG. 5B, at least some (e.g., 30-99%, 30-75%) a majority (e.g., 50% or more), most (e.g., 75-99%), or all of the wells 522 may be connected to at least one other well by at least one channel, pore, hole, and the like. The channels, pores, holes can have a size (e.g., opening and/or inner diameter) that is less than the size of the opening of the wells. For example, the size of the channels, pores, holes can be 30-60 micrometers (e.g., 35-55, 40-50 micrometers). After implantation, cells/tissue can migrate into the wells 522 and through the channels, pores, holes and deposit connective tissue proteins. The connective tissue proteins can become cross-linked over time such that the electrode becomes integrated with the tissue (e.g., a tissue-to-electrode interface). As such, the micropores can be connected to one or more other micropores by channels.

In the example of FIG. 5B, a microporous surface texture 54 is provided on a face electrode or a face portion of an end electrode (e.g., the face electrode 411 or the face electrode portion 34 (FIGS. 3A-3B) of the end electrode of FIGS. 2A and 4). The microporous surface texture 54 includes an array of wells 542 in a mesh-like structure 544. In some instances, the wells 542 may have a configuration (e.g., a dimension, a shape, a material, and the like) similar to the wells 522 of FIG. 5A. In some instances, the wells 542 have a configuration (e.g., a dimension, a shape, a material, and the like) that is different from that of the wells 522 such that the porous surface textures 54 and 52 have different tissue in-growth rates.

Some, a majority, most, or all of the wells 522 can be connected to at least one other well 522 by at least one channel, pore, hole, and the like. In FIG. 5B, the channel, pore, hole, etc. are represented by reference number 53 and are hereinafter referred to as the channels 53 for brevity. In certain instances, the channels 53 have an opening or diameter of 30-60 micrometers. After implantation, the channels 53 allow cells/tissue to migrate into the wells 522 and through the channels 53—which results in deposition of extracellular matrix proteins such that the electrode becomes integrated with and attached to the tissue. In certain instances, the channels 53 extend through a wall of the wells 522. In other instances, the channels 53 extend between the respective base of two of the wells 522. The electrodes can include micropores and channels extending between a given set of micropores regardless of the whether the electrodes (or portion thereof) are on a face of the IMD or an end of the IMD. Put another way, both face electrodes and end electrodes can include a microporous surface area with channels extending between micropores within the electrode.

In the example of FIG. 5C, a microporous surface texture 56 is provided on a face electrode or a face portion of an end electrode. The face electrode or the face portion of the end electrode has a dome shape with the porous surface texture 56 formed thereon. As shown, the porous surface texture 56 includes an array of circular-shaped wells 562 formed into the dome surface 55. In certain instances, the wells 562 can have a curved surface (e.g., similar to a dimples on a golf ball), while in other instances, the wells 562 are cylinder-shaped. The wells 562 can include channels 57 that extend between respective wells 562—similar to the channels 53 described above.

In the example of FIG. 5D, a porous surface texture 58 is provided on the outer surface of an end electrode (e.g., the end electrodes of FIGS. 2A and 4). The porous surface texture 58 includes a random surface pattern of granular structures or islands which may be connected to each other. It is to be understood that the random surface pattern of the porous surface texture 58 can be formed or superimposed on any porous surface textures described herein such as, for example, the porous surface texture 52 of FIG. 5A, the porous surface texture 54 of FIG. 5B, and the porous surface texture 56 of FIG. 5C. For example, a random surface pattern can be formed on a bottom surface of a well. Like the examples above, channels can be formed to extend between different portions of the porous surface texture.

In the example of FIG. 5E, a porous surface texture 62 is provided on a portion of the curved edge electrode portion 32 of an end electrode (e.g., the end electrode 211, 221 or 311). The porous surface texture 62 includes an array of microstructures 622, which are partially overlapped with to connected to adjacent microstructures. Like the examples above, channels can be formed to extend between different portions of the porous surface texture.

In the example of FIG. 5F, a porous surface texture 64 is provided on the outer surface of an end electrode (e.g., the end electrode 211, 221 or 311). The porous surface texture 64 includes an array of pits 642. The adjacent pits may or may not be partially overlapped with other. Like the examples above, channels can be formed to extend between different portions of the porous surface texture.

In some instances, one or more microporous surface areas can be provided on a surrounding, electrically non-conductive area adjacent to an electrode on an outer surface of an IMD. The surrounding, electrically non-conductive area can be, for example, a polymer surface. Referring back to FIG. 2A, a first microporous surface area 722 is formed on an outer surface of the first housing section 210 adjacent to the first end electrode 211, and a second microporous surface area 724 is formed on an outer surface of the second housing section 220 adjacent to the second end electrode 221. Each of the first microporous surface area 722 and the second microporous surface area 724 can form a ring shape having one edge 73 in contact to an inner end 74 of the respective end electrodes 211 and 221. Each of the first microporous surface area 722 and the second microporous surface area 724 can include a microporous surface texture described herein (e.g., the microporous surface texture 52, 54, 56, 58, 62 and 64 in FIGS. 5A-5F). Similarly, a microporous surface area including a microporous surface texture can be provided on a surrounding, electrically non-conductive area 726 adjacent to the face electrode 411 as shown in FIG. 4. The microporous surface area 726 can be formed on the major surface 212 of the first housing section 210, at least partially surrounding the face electrode 411.

In some instances, a first microporous surface texture or microporous features can be formed on a first microporous surface area of an electrode, and a second microporous surface texture or microporous features can be formed on a second microporous surface area on a surrounding, electrically non-conductive area (e.g., a polymer surface) adjacent to the electrode. The first microporous surface texture or microporous feature can have a first characteristic dimension. The second microporous surface texture or microporous feature can have a second characteristic dimension which may be different from the first characteristic dimension.

In some instances, one or more protrusions can be placed on the outer surface of the IMD to reduce movement of the IMD within an implantation location or pocket in the patient's chest or abdomen. The protrusions can provide a structure that reduces at least one of rotational, translational, and lateral movement of the IMD within a patient's tissue after implantation in the patient. For example, having protrusions arranged at least partially across the width of an IMD may prevent longitudinal movement of the IMD within a patient's tissue, and having protrusions arranged at an angle to the central axis of the IMD may prevent the IMD from flipping or rotating within the patient's tissue. Additionally, having protrusions attached to the outer surface of the IMD may provide a structure that is complementary to the teeth of an insertion or extraction tool, giving a medical provider more control over the IMD while being held. For example, the protrusions may be sized and shaped to provide added frictional engagement with a medical forceps. The protrusions may make it easier for a health care worker to grab the IMD with an extraction tool and remove the IMD from a patient without the IMD slipping from the extraction tool.

As shown in FIG. 2A, an array of protrusions 82 is provided on the major surface 212 of the first housing section 210. As shown in FIG. 4, a first set of protrusions 84A is provided on the major surface 212 of the first housing section 210 adjacent to the fourth housing section 240, and a second set of 84B is provided on the major surface 212 of the first housing section 210 adjacent to the first end 202. The protrusions 82, 84A, and 84B may have various shapes such as, for example, a truncated triangular prism shape, an elongated truncated pyramid shape, and the like.

One or more of the protrusions can include one or more holes or channels that extend through a given protrusion 82. For example, the channels can have a diameter of 30-200 micrometers (e.g., 30-60, 50-150, 100-200 micrometers). The channels can extend between one side of the protrusions to another side of the protrusions along a longitudinal direction/axis of the IMD. Like the channels in the electrodes described above, the channels in the protrusions can encourage cells/tissue to deposit and interconnect through the channels.

FIG. 6 a side view of a portion of an IMD 300 being gripped by a portion of a medical forceps 302, in accordance with some instances of the disclosure. The IMD 300 may be, be identical to, or be similar to, the IMD 102 depicted in FIG. 1, the IMD 200 depicted in FIG. 2A, and/or the IMD 300 depicted in FIG. 4. As shown, for example, in FIG. 6, the IMD 300 includes a header 304 having a first end 306 and a second end 308. The second end 308 of the header 304 is coupled, via a feed-through assembly 310 to a core assembly 312. As shown in FIG. 6, the IMD 300 includes an outer surface 314, which includes a first surface 316 and a second, opposite and parallel, surface 318.

As shown, a first set 320 of protrusions 322 is disposed on the first surface 316 near the second end 308 of the header 304; a second set 324 of protrusions 326 is disposed on the first surface 316 near the first end 306 of the header; a third set 328 of protrusions 330 is disposed on the second surface 318 near the second end 308 of the header; and a fourth set 332 of protrusions 334 is disposed on the second surface 318 near the first end 306 of the header 304. As shown, the protrusions 322, 326, 330, and 334 are shaped and arranged to correspond to the shape and arrangement, respectively, of the teeth 336 of the medical forceps 302. For example, the width of each protrusion may be sized to fit within each of the spaces in the grips of a medical forceps; and the height of each protrusion may be sized to be received a distance into the grip of a medical forceps. In some instances, the protrusions 322, 326, 330, and 334 may be sized with a complementary height, length, and/or width to the teeth 336 of a standard medical forceps, a custom medical forceps, and/or the like. In instances, the protrusions 322, 326, 330, and 334 may be configured in different sizes so that at least one of the protrusions 322, 326, 330, and 334 corresponds to one of several different styles, sizes, and/or shapes of forceps. In instances, for example, the protrusions maybe shaped to correspond to a medical forceps having a grid patterned grip, a checkered grip, a number of rows of teeth, and/or any suitable pattern for gripping or holding.

### Methods

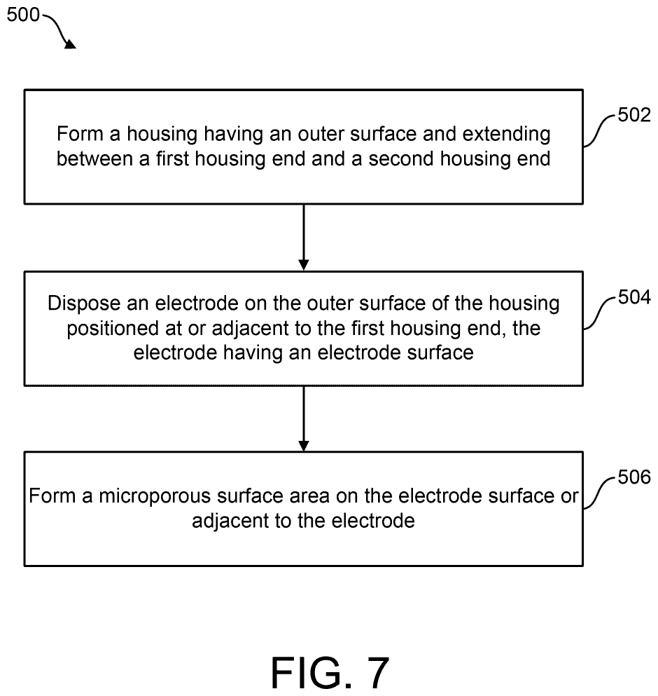

FIG. 7 shows a block diagram of a method 500 of manufacturing the IMDs described above. The method 500 includes forming a housing having an outer surface and extending between a first housing end and a second housing end (block 502).

The method 500 further includes disposing an electrode on the outer surface of the housing positioned at or adjacent to the first housing end (block 504). The electrode has an electrode surface. The method 500 further includes forming a microporous surface area on the electrode surface or adjacent to the electrode (block 506). The microporous surface area includes microporous surface textures (e.g., a collection or set of micropores) configured to allow a tissue in-growth on the microporous surface area after implantation of the implantable medical device within a patient's tissue. The electrodes can include channels that extend between the micropores such that cells/tissue can integrate with the electrodes to create a tissue-to-electrode interface. In some instances, a first microporous feature or microporous surface texture is formed on the electrode surface. In some instances, a second microporous feature or microporous surface texture is formed at least partially surrounding the electrode. In certain instances, the microporous surface area is formed on the electrode before the electrode is coupled to the housing, while in other instances, the electrode is coupled to the housing before the microporous surface area is formed on the electrode.

In some instances, the method 500 further includes forming a plurality of protrusions disposed on the outer surface of the housing. In some instances, the plurality of protrusions are configured to reduce at least one of rotational, translational, and lateral movement of the implantable medical device within a patient's tissue after implantation in the patient. One or more of the protrusions can include at least one channel that is formed through a given protrusion.

Various modifications and additions can be made to the exemplary instances discussed without departing from the scope of the disclosed subject matter. For example, while the instances described above refer to particular features, the scope of this disclosure also includes instances having different combinations of features and instances that do not include all of the described features. Accordingly, the scope of the disclosed subject matter is intended to embrace all such alternatives, modifications, and variations as fall within the scope of the claims, together with all equivalents thereof.

We claim:

1. An implantable medical device (IMD) comprising:
a housing having an outer surface and extending between a first housing end and a second housing end;
an electrode coupled to the housing and positioned at or adjacent to the first housing end, the electrode having an electrode surface; and
a surface area on the electrode surface or adjacent to the electrode,
wherein the surface area includes micropores and channels interconnecting the micropores.

2. The IMD of claim 1, wherein the surface area is on the electrode surface.

3. The IMD of claim 2, wherein the electrode is a face electrode.

4. The IMD of claim 2, wherein the electrode is an end electrode.

5. The IMD of claim 1, wherein the electrode is a first electrode, the IMD further comprising a second electrode that includes a second set of micropores and a second set of channels interconnecting the second set of micropores.

6. The IMD of claim 5, wherein the first electrode is an end electrode, wherein the second electrode is a face electrode.

7. The IMD of claim 6, wherein the first electrode is a first end electrode, wherein the second electrode is a second electrode.

8. The IMD of claim 1, wherein the micropores include a first micropore and a second micropore adjacent to each other, wherein one of the channels extends between the first micropore and the second micropore.

9. The IMD of claim 1, wherein the micropores have an opening that is 50-200 micrometers.

10. The IMD of claim 1, wherein the micropores have a depth of 100-500 micrometers.

11. The IMD of claim 1, wherein the channels have an opening of 30-60 micrometers.

12. The IMD of claim 1, wherein the electrode is directly coupled to the housing.

13. The IMD of claim 1, wherein the micropores have circular-shaped openings.

14. The IMD of claim 1, wherein the micropores have square-shaped openings.

15. The IMD of claim 1, further comprising protrusions disposed on the outer surface of the housing, wherein the protrusions have holes extending through the protrusions.

16. The IMD of claim 1, wherein a majority of the micropores are connected to at least one other micropore via at least one of the channels.

17. The IMD of claim 1, wherein the micropores and the channels are shaped and sized for deposition of extracellular matrix proteins.

18. An implantable medical device (IMD) comprising:
a housing having an outer surface and extending between a first housing end and a second housing end;
an electrode coupled to the housing and positioned at or adjacent to the first housing end, the electrode having an electrode surface; and
a surface area on the electrode surface or adjacent to the electrode,
wherein the surface area includes micropores and channels interconnecting the micropores, wherein the micropores have respective openings that are 50-200 micrometers, wherein the micropores have respective depths of 100-500 micrometers, wherein the channels have respective diameters of 30-60 micrometers.

19. A method of making an implantable medical device (IMD), the method comprising:
forming a housing having an outer surface and extending between a first housing end and a second housing end;
disposing an electrode on the outer surface of the housing; and
forming a microporous surface area on the electrode or adjacent to the electrode,
wherein the microporous surface area includes micropores and channels interconnecting the micropores.

20. The method of claim 19, wherein the micropores include a first micropore and a second micropore adjacent to each other, wherein one of the channels extends between the first micropore and the second micropore.
